SEQUENCE LISTING (50 sequences)

SEQ ID NO: 1 - protein, 21 aa, Homo Sapiens
  CHAIN (1)..(21)
ARDPRYYDAWSGPQLYYYMDV

SEQ ID NO: 2 - protein, 9 aa, Homo Sapiens
  CHAIN (1)..(9)
GFSLSTYAT

SEQ ID NO: 3 - protein, 8 aa, Homo Sapiens
  CHAIN (1)..(8)
ISHNGNIE

SEQ ID NO: 4 - protein, 13 aa, Homo Sapiens
  CHAIN (1)..(13)
SGSSPNIGSNSAH

SEQ ID NO: 5 - protein, 7 aa, Homo Sapiens
  CHAIN (1)..(7)
MNIERPS

SEQ ID NO: 6 - protein, 11 aa, Human Immunodeficiency virus type
  CHAIN (1)..(11)
ELWGGPKLTVP

SEQ ID NO: 7 - protein, 35 aa, Homo Sapiens
  CHAIN (1)..(35)
SQTQQEKNEQELLELDKWASLWNWFDITNWLWYIK

SEQ ID NO: 8 - protein, 226 aa, Homo Sapiens
  CHAIN (1)..(226)
ESGGGVVQPGRSLRLSCAASGFSLSTYAIHWVRQAPGKGLEWVAVISHNGNIEYFGDSVRGRFSISRDNSKNTVYLHMNSLTTEDTAVYYCARDPRYYDAWSGPQLYYYYMDVWGKGTTVTVSSASPTSPKVFPLSLDSTQPDGNVVIACLVQGFFPQEPLSVTWSESGQGVTARDFPPSQDASGDLYTTSSQLTLPATQCLAGKSVTCHVKHYTNPSQDVTVPCP

SEQ ID NO: 9 - protein, 206 aa, Homo Sapiens
  CHAIN (1)..(206)
QSVLTQPPSASGTPGQRDTISCSGSSPNIGSNSAHWHQRVPGTAPKLLIYMNIERPSGVPDRISGSKSDSSASLAISGLRSGDEVDYFCELWGGPKLTVPGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - DNA, 678 bp, Homo Sapiens
  misc_feature (1)..(678)
gagtctgggggaggcgtcgtccagcctgggcggtccctgagactctcctgtgcagcctctggcttcagcctcagtacgtatgctatacactgggtccgccaggctccaggcaagggactggagtgggtggctgttatttctcacaacggcaacattgaatacttcggagactccgtgaggggacgattcagcatctccagagataattccaaaaacacagtgtatctccatatgaacagcctgacaactgaggacacggctgtctattattgtgcgagagatcctcggtattacgatgcttggagcggcccccaactttactactactacatggacgtctggggcaaagggaccacggtcaccgtctcctcagcatccccgaccagccccaaggtcttcccgctgagcctcgacagcacccagccagatgggaacgtggtcatcgcctgcctggtccagggcttcttcccccaggagccactcagtgtgacctggagcgaaagcggacagggcgtgaccgccagagacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgccca

SEQ ID NO: 11 - DNA, 618 bp, Homo Sapiens
  misc_feature (1)..(618)
cagtctgtgctgactcagccaccctcagcgtctgggacccccgggcagagggacaccatctcttgttctggaagcagtcccaacatcggaagtaattctgctcactggcaccagcgggtcccaggaacggcccccaaactcctcatctatatgaatattgagcggccctcaggggtccctgaccgaatctctggctccaagtctgactcctcagcctccctggccatcagtgggctccggtccggggatgaagttgattatttctgtgagttgtggggggggcccaagctgaccgtcccaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 12 - protein, 27 aa, Homo Sapiens
  DISULFID (1)..(25)
CARDPRYYDAWSGPQLYYYYMDVWCKG

SEQ ID NO: 13 - DNA, 24 bp, Artificial Sequence
  Primer
caggtgcagctcgagcagtctggg

SEQ ID NO: 14 - DNA, 24 bp, Artificial Sequence
  Primer
caggtgcagctgctcgagtctggg

SEQ ID NO: 15 - DNA, 24 bp, Artificial Sequence
  Primer
caggtcaccttgctcgagtctggt

SEQ ID NO: 16 - DNA, 24 bp, Artificial Sequence
  Primer
gaggtgcagctcgaggagtctggg

SEQ ID NO: 17 - DNA, 24 bp, Artificial Sequence
  Primer
gaggtgcagctgctcgagtctggg

SEQ ID NO: 18 - DNA, 23 bp, Artificial Sequence
  Primer
caggtgcagctgctcgagtcggg

SEQ ID NO: 19 - DNA, 23 bp, Artificial Sequence
  Primer
caggtgcagctactcgagtgggg

SEQ ID NO: 20 - DNA, 23 bp, Artificial Sequence
  Primer
gaggtgcagctcgagcagtctgg

SEQ ID NO: 21 - DNA, 23 bp, Artificial Sequence
  Primer
caggtacagctcgagcagtcagg

SEQ ID NO: 22 - DNA, 23 bp, Artificial Sequence
  Primer
caggtgcagctcgagcaatctgg

SEQ ID NO: 23 - DNA, 30 bp, Artificial Sequence
  Primer
agttgaactagttgggcagggcacagtcac

SEQ ID NO: 24 - DNA, 30 bp, Artificial Sequence
  Primer
agttgaactagttcggcagggaacagtcac

SEQ ID NO: 25 - DNA, 24 bp, Artificial Sequence
  Primer
gacatcgagctcacccagtctcca

SEQ ID NO: 26 - DNA, 23 bp, Artificial Sequence
  Primer
gacatcgagctcacccagtctcc

SEQ ID NO: 27 - DNA, 24 bp, Artificial Sequence
  Primer
gatattgagctcactcagtctcca

SEQ ID NO: 28 - DNA, 18 bp, Artificial Sequence
  Primer
gatattacccagactcca

SEQ ID NO: 29 - DNA, 24 bp, Artificial Sequence
  Primer
gaaattgagctcacgcagtctcca

SEQ ID NO: 30 - DNA, 24 bp, Artificial Sequence
  Primer
gaaattgagctcacncagtctcca

SEQ ID NO: 31 - DNA, 18 bp, Artificial Sequence
  Primer
gacatcacccagtctcca

SEQ ID NO: 32 - DNA, 24 bp, Artificial Sequence
  Primer
gaaacggagctcacgcagtctcca

SEQ ID NO: 33 - DNA, 24 bp, Artificial Sequence
  Primer
gaaattgagctcactcagtctcca

SEQ ID NO: 34 - DNA, 26 bp, Artificial Sequence
  Primer
cagtctgagctcacgcagccnccctc

SEQ ID NO: 35 - DNA, 26 bp, Artificial Sequence
  Primer
cagtctgagctcactcagcctgcctc

SEQ ID NO: 36 - DNA, 24 bp, Artificial Sequence
  Primer
gcctcctatgagctcactcagcca

SEQ ID NO: 37 - DNA, 26 bp, Artificial Sequence
  Primer
cagcctgagctcactcaatcatcctc

SEQ ID NO: 38 - DNA, 26 bp, Artificial Sequence
  Primer
cagcctgagctcactcagcccccgtc

SEQ ID NO: 39 - DNA, 27 bp, Artificial Sequence
  Primer
cagcctgagctcactcagccgncttcc

SEQ ID NO: 40 - DNA, 24 bp, Artificial Sequence
  Primer
aattttgagctcactcagccccac

SEQ ID NO: 41 - DNA, 24 bp, Artificial Sequence
  Primer
cagactgagctcactcaggagccc

SEQ ID NO: 42 - DNA, 24 bp, Artificial Sequence
  Primer
cagactacccaggagccatcgttc

SEQ ID NO: 43 - DNA, 26 bp, Artificial Sequence
  Primer
cagcctgagctcactcagccaccttc

SEQ ID NO: 44 - DNA, 27 bp, Artificial Sequence
  Primer
caggcagagctcactcagccaccctcg

SEQ ID NO: 45 - DNA, 36 bp, Artificial Sequence
  Primer
tccttctagattactaacactctcccctgttgaagc

SEQ ID NO: 46 - DNA, 30 bp, Artificial Sequence
  Primer
cgccgtctagaattatgaacattctgtagg

SEQ ID NO: 47 - DNA, 22 bp, Artificial Sequence
  Primer
aagacagctatcgcgattgcag

SEQ ID NO: 48 - DNA, 21 bp, Artificial Sequence
  Primer
acctattgcctacggcagccg

SEQ ID NO: 49 - DNA, 22 bp, Artificial Sequence
  Primer
gagcagctgcacctcggccatg

SEQ ID NO: 50 - DNA, 24 bp, Artificial Sequence
  Primer
gcccccttattagcgtttgccatc

(19) United States
(12) Patent Application Publication
(10) Pub. No.: US 2009/0191216 A1
(43) Pub. Date: Jul. 30, 2009

(54) Antibody or a fragment thereof, having neutralizing activity against HIV

(75) Inventors: Morgane Bomsel, Paris (FR); Daniela Tudor, Fontenay Aux Roses (FR)

Correspondence Address: OLIFF & BERRIDGE, PLC, P.O. BOX 320850, ALEXANDRIA, VA, 22320-4850, US

(73) Assignee: INSTITUT NATIONAL DE LA SANTE ET DE LA RECHERCHE MEDICALE, PARIS (FR)

(21) Appl. No.: 11/979,326

(22) Filed: Nov. 1, 2007

Related U.S. Application Data
(63) Continuation-in-part of application No. PCT/IB2005/001180, filed on May 2, 2005.

(51) Int. Cl. A61K 39/42; C07K 16/00; C07K 16/10; C07K 14/00; C12N 15/11; C12N 15/00; A61K 38/16

(52) U.S. Cl. 424/148.1; 530/388.2; 530/388.15; 530/388.35; 530/317; 536/23.53; 435/320.1; 514/9

(57) ABSTRACT

The invention relates to a monoclonal antibody or a fragment thereof, recognizing a peptide of sequence set forth as SEQ ID NO 7 or an analogue thereof, wherein the complementarity determining region 3 (CDR3) of its H chain variable region comprises the peptide sequence set forth as SEQ ID NO 1 or a functional analogue thereof.

The instant invention relates to an antibody or a fragment thereof and to a cyclized peptide, having neutralizing activity against HIV, and useful in the treatment and/or prevention of HIV infection. The invention also relates to a pharmaceutical composition comprising said antibody or cyclized peptide, a diagnostic composition and a method for providing passive immunotherapy.

Human immunodeficiency virus (HIV) is a member of the lentivirus family of animal retroviruses and is a causative agent of Acquired Immune Deficiency Syndrome (AIDS). To date, two closely related types of HIV type 1 (HIV-1) and type 2 (HIV-2) have been identified and characterized at the molecular level.

The introduction of antiviral agents against HIV, such as reverse transcriptase inhibitors, has allowed to greatly improve the conditions of the patient affected by AIDS. However, in most cases, therapeutic efficacy of these drugs to AIDS is partial or temporal, and in addition, these drugs exhibit toxicity or growth inhibition to haematopoietic cells, and thereby inhibit reconstruction of an immune system which has become deficient.

Therefore, it is generally agreed that AIDS prevention programs and antiretroviral drug therapies (VALDISERRI, 2003, Nat. Med, 9:881) should be combined with effective microbicides and vaccines. But the design and testing of such vaccines have proven to be complex (LETVIN, et al. 2002, Annu. Rev. Immunol., 20:73; McMICHAEL & HANKE, 2003, Nat. Med., 9:874).

Mucosal surfaces are the major site for HIV-1 entry (NICOLOSI, et al. 1994, J. Acquir. Immun. Defic. Syndr., 7:296). HIV-1 transmission may occur through exposure of mucosal surfaces to HIV-1 infected fluids, such as semen, colostrums, breast milk, and cervico-vaginal fluid (CHERMANN, 1998, Am. J. Reprod. Immunol., 40:183; MILMAN & SCHARMA, 1994, AIDS, 10:1305).

Because of the interaction of HIV with mucosal surface, a component of a vaccine against HIV should engage the mucosal immune system to interfere with early steps of mucosal viral transmission and potential receptors.

It has been recognized that antibodies neutralizing AIDS viruses may clearly play an important role in protection.

However, while it was possible to achieve neutralizing antibodies against a specific virus strain grown in the laboratory, the carrying out of antibodies that could neutralized a broad array of strains and that could be effectively active in vivo has not yet met similar success.

Therefore, there is a need for an antibody that allows for neutralizing and/or mitigating HIV infection, and in particular HIV-1 infection.

There is a need for an antibody that allows to prevent and/or reduce the HIV infection through mucosal surface.

There is also a need for providing an antibody for the manufacture of a medicament intended to be used in passive immunotherapy.

There is a need for an antibody that may be used for diagnostic purposes.

The invention has for object to satisfy to all or part of those above-mentioned needs.

The inventors have obtained an efficient monoclonal antibody that allows to satisfy the above-mentioned unmet needs. In particular, the inventors have identified a novel secretory IgA (S-IgA) antibody Fab having a specific complementarity determining region 3 (CDR3) of the H chain variable region, said antibody recognizing a specific peptide from the gp41 protein of HIV-1. The newly identified antibody has also the ability to inhibit HIV-1 transcytosis and block the CD4+T cells infection.

Therefore, according to one of its first aspects, the instant invention is related to a monoclonal antibody or a fragment thereof, recognizing a peptide of sequence set forth as SEQ ID NO:7 or an analogue thereof, wherein the complementarity determining region 3 (CDR3) of its H chain variable region comprises the peptide sequence set forth as SEQ ID NO:1 or a functional analogue thereof.

The terms “antibody fragment” as used herein refers to an antibody that has an amino-terminal and/or carboxy-terminal deletion but where the remaining aminoacid sequence is identical to the corresponding positions in the naturally occurring sequence and which has its biological properties maintained or non adversely affected. This antibody fragment may comprise additional modifications such as insertion, deletion and/or substitution of aminoacid residues and/or fusion with other peptides or proteins to make chimeric proteins. The term “antibody fragment” may also encompass the various parts of an antibody, i.e. the constant, variable, heavy and light chains.

Within the meaning of the invention, the expression “functional analogue” with respect to a peptide is intended to refer to a peptide that has amino acid sequence homology or identity with another amino acid sequence and that has similar or conserved biological properties relative to said other peptide sequence. Typically, peptide analogues comprised conservative amino acid substitutions (and/or insertions and/or deletions) with respect to the naturally-occurring sequence.

According to another of its aspects, the instant invention is directed to a H chain variable region recognizing a peptide sequence set forth as SEQ ID NO:7 or an analogue thereof, wherein the CDR3 of said H chain variable region comprises the peptide sequence set forth as SEQ ID NO:1 or a functional analogue thereof.

According to another of its aspects, the instant invention is directed to a cyclized peptide having from 24 to 40 amino acid residues and comprising a loop, said loop comprising a peptide of sequence set forth as SEQ ID NO:1, or a functional analogue thereof, said cyclized peptide recognizing a peptide of sequence set forth as SEQ ID NO:7 or a functional analogue thereof.

According to another of its aspects, the antibodies and the cyclized peptides of the invention or fragments thereof have the ability to neutralize HIV.

According to another of its aspects, the instant invention also concerns nucleic acid sequence encoding for the antibody of the invention or a fragment thereof or the cyclized peptides of the invention, as well as a vector and host cell comprising said nucleic acid sequence.

According to another of its aspects, the instant invention relates to a pharmaceutical composition comprising as active agent an effective amount of an agent chosen among an antibody of the invention or a fragment thereof or a cyclized peptide of the invention, a nucleic acid sequence of the invention, a vector of the invention or a host cell of the invention, and a suitable carrier.

According to another of its aspects, the instant invention is also directed to a diagnostic composition and a method for detecting, in vitro, a HIV strain in a sample.

According to another of its aspects, the instant invention also concerns a method for providing passive immunotherapy to an individual liable to be infected with HIV comprising administering a therapeutically effective amount of at least an antibody of the invention or a fragment thereof or a cyclized peptide of the invention.

Antibodies

The antibody according to the invention refers to an intact immunoglobulin, or a fragment thereof, and in particular to an antigen binding portion thereof.

An immunoglobulin is a tetrameric molecule, composed of two identical pairs of polypeptide chains, each pair having one “light” (L) (about 25 kDA) and one “heavy” (H) chain (about 50-70 kDa). The amino-terminal portion of each chain includes a variable region (V) of about 100 to 110 or more amino acids, primarily responsible for antigen recognition. The carboxy-terminal portion of each chain defines a constant region (C) primarily responsible for effector function. Human light chains are classified as λ and κ light chains. Heavy chain constant regions are classified as μ, δ, γ, α, or ε, and define the antibodies isotype as IgM, IgD, IgG, IgA and IgE respectively. The variable regions of each light/heavy chain pair form the antibody binding site, such that an intact immunoglobulin generally has at least two binding site.

In a particular embodiment of the invention, the monoclonal antibody of the invention or fragment thereof, may be an IgA, and more particularly a secretory IgA (S-IgA).

According to another embodiment, the monoclonal antibody of the invention, or a fragment thereof may be a human antibody.

Immunogobulin chains display the same general structure of relatively conserved framework regions (FR) joint by free hypervariable regions also called complementarity determining regions or CDRs. The CDRs from the two chains of each pair are aligned by the framework regions, enabling binding to a specific epitope (antigen-binding portion of the antibody). From N-terminus to C-terminus, both light and heavy chains comprise the domains FR1, CDR1, FR2, CRD2, FR3, CDR3 and FR4.

The antibody of the invention comprises notably as CDR3 of the H chain variable region, the peptide sequence set forth as SEQ ID NO:1 or a functional analogue thereof. This sequence is 21 amino acids in length and comprises in the middle an aromatic hydrophobic amino acid residue such as a tryptophan or a phenylalanin (W or F) (10^{th} amino acid starting from the N-terminus, identified as a W in the one letter amino acid code in SEQ ID NO:1).

Without being bound by any particular theory, the inventors consider that this aromatic hydrophobic amino acid residue may play a critical role in the recognition of the antigen, i.e. the peptide P1 identified by the peptide sequence set forth as SEQ ID NO:7. Therefore, it is considered that any functional analogue of the CDR3 sequence of the invention should have a length of sequence of at least about 15 amino acids, preferably of at least about 18 amino acids, and more particularly of about 20-21 amino acids and should comprise in the middle of its sequence an aromatic hydrophobic residue such as a tryptophan or a phenylalanine or an analogue thereof.

According to another embodiment, the H chain variable region of the monoclonal antibody of the invention or a fragment thereof further comprises at least one CDR chosen among CDR1 and CDR2 having respectively the peptide sequence set forth as SEQ ID NO:2 and SEQ ID NO:3 or functional analogues thereof.

According to a particular embodiment, the H chain variable region of the monoclonal antibody of the invention or a fragment thereof comprises as CDRs, the CDR1, CDR2 and CDR3 having respectively the peptide sequence set forth as SEQ ID NO:2 and SEQ ID NO:3 and SEQ ID NO:1.

Within another embodiment, the invention is also directed to the H chain variable region as previously defined.

The H chain variable region of the invention may recognize a peptide called P1 and having the peptide sequence set forth as SEQ ID NO:7 or an analogue thereof.

The peptide P1 corresponds to an amino acid sequence present in the HIV envelope protein gp41 that is exposed at the surface of the viral particles before the viruses interact with target cells. In HIV-1 strain, this sequence is comprised from amino acid 650 to amino acid 685 of the gp41 protein.

In solution, this peptide may adopt a structured, concentration dependent oligomeric state, namely dimeric or tetrameric state (ALFSEN & BOMSEL, 2002, J. Biol. Chem., 277: 25649).

Therefore, an antibody or a H chain variable region according to the invention or fragment thereof may recognize the peptide P1 under a monomeric or an oligomeric form.

The peptide sequence of P1 (SEQ ID NO:7) comprises various epitope sequences recognized by various antibodies known in the art. Such epitopes are the epitope ELDKWA recognized by the monoclonal antibody IgG 2F5 and the epitope NWFDIT recognized by the monoclonal antibody IgG 4E10.

Within an embodiment of the invention, the antibody of the invention or fragment thereof does not recognize, the epitopes known as 2F5 and 4E10.

Within another embodiment of the instant invention, the antibody according to the invention or a fragment thereof, may also comprise a L chain variable region comprising at least one complementarity determining region chosen among CDR1, CDR2 and CDR3 having respectively the peptide sequence set forth as SEQ ID NO:4, SEQ ID NO:5 and SEQ ID NO:6 or functional analogues thereof.

Therefore, according to another embodiment, the instant invention relates to a L chain variable region comprising at least one CDR chosen among CDR1, CDR2 and CDR3, having respectively the peptide sequence set forth as SEQ ID NO:4, SEQ ID NO:5 and SEQ ID NO:6 or functional analogues thereof.

According to another embodiment, the L chain variable region of the invention comprises as CDRs the CDR1, CDR2 and CDR3 having respectively the peptide sequence set forth as SEQ ID NO:4, SEQ ID NO:5 and/or SEQ ID NO:6.

According to another embodiment, the monoclonal antibody according to the invention or a fragment thereof comprises the H chain variable region and the L chain variable region having respectively the amino acid sequence set forth as SEQ ID NO:8 and SEQ ID NO:9.

According to an embodiment, the antibody according to the invention is a Fab identified as clone 43 in the experimental section and comprising the H chain variable region and the L chain variable region having respectively the amino acid sequence set forth as SEQ ID NO:8 and SEQ ID NO:9.

According to another embodiment, the antibody of the invention may be a recombinant anti-HIV antibody or a fragment thereof, comprising a H chain variable region of the invention.

According to another embodiment, the recombinant antibody of the invention may comprise, additionally, the L chain variable region of the invention.

Hence, the CDR3 of the H chain variable region of the invention, or the H chain variable region as a whole, may be used to construct chimeric or recombinant antibody having a framework distinct of the IgA isotype, such as for example a framework of IgG isotype. Such construction may be obtained by any molecular biology tools known in the art, such as described in “Molecular Cloning—A Laboratory Manual” (2^{nd} ed.), Sambrook et al., 1989, Coldspring Harbor Laboratory, Coldspring Harbor Press, New York (Sambrook). The chimeric or recombinant antibody may be a whole antibody or a fragment thereof.

Accordingly, a functional analogue of a CDR of the invention of the H chain variable region or of the L chain variable region, once inserted in lieu of the original sequence in an antibody according to the invention, maintain its ability or does not adversely affect its ability to recognize the peptide of SEQ ID NO:7, or a functional analogue thereof, and its ability to neutralize HIV and/or inhibit HIV infection.

The present invention also relates to antibodies comprising a Fab fragment derived from a human antibody of this invention and the human Fc domain derived from an other Ig subtype, such as IgG and the like.

Therefore, according to an embodiment, the invention is also directed to an antigen binding portion of an antibody according to the invention or a fragment thereof, comprising a H chain variable region as previously defined. This antigen binding portion may optionally comprise a L chain variable region as previously defined.

Antigen-binding portions may be produced by any known recombinant DNA techniques or by enzymatic or chemical cleavage of intact antibodies. Antigen-binding portions may include, inter alia, Fab (monovalent fragment consisting of the VL, VH, CL and CHI domains), F(ab′)₂ (bivalent fragment comprising two Fab fragments linked by a disulfide bridge at the hinge region), Fv (fragment consisting of the VL and VH domains of a single arm of an antibody), Fd (fragment consisting of the VH and CH1 domains), dAb fragment (consisting of a VH domain), scFv (consisting of an antibody in which VL and VH regions are paired to form a monovalent molecules via a synthetic linker), chimeric antibody, diabodies, and complementarity determining region (CDR) fragments.

Cyclized Peptides

According to another of its aspects, the invention also relates to cyclized peptide prepared by cyclization of a CDR3 of a H chain variable region of the invention or a functional analogue thereof.

Therefore, according to one exemplary embodiment, the instant invention is related to a cyclized peptide, and in a particular to a cyclized peptide having from 24 to 40 amino acid residues and comprising a loop, said loop comprising a peptide of sequence set forth as SEQ ID NO:1, or a functional analogue thereof, said cyclized peptide recognizing a peptide of sequence set forth as SEQ ID NO:7 or a functional analogue thereof.

The instant invention is also related to sequences of cyclized peptides that are to be cyclized, that is which are linearly provided

Within the meaning of the invention, the term “loop” within a context of a cyclized peptide is intended to mean all or part of the amino acid sequence of said peptide present into a cycle formed by bridging at least two amino acid residues comprised in the amino acid sequence of said peptide.

Such cyclized peptide may comprise additional amino acid residues flanking the amino acid sequence corresponding to a CDR3 of a H chain variable region of the invention or a functional analogue thereof.

A cyclized peptide in accordance with the invention may comprise at least one, in particular 2, in particular 3, more particularly 6, and more particularly 8 additional amino acids at one or both extremities of the sequence of a CDR3 of a H chain variable region of the invention or a functional analogue thereof.

An additional amino acid may be present inside or outside the loop.

An additional amino acid may be an amino acid naturally occurring in a wild-type sequence of a H chain variable region flanking a CDR3 of the invention or may result from a natural or artificial mutation (addition, substitution or insertion), for example resulting from any known techniques in the art related to recombinant DNA or by any known techniques of chemical synthesis.

The cyclization of a CDR3 in accordance with the invention may be performed by any known techniques in the art, as for example described in LEVI et al (Proc. Natl. Acad. Sci. USA. 1993, 90:4374).

As example of technique of cyclization suitable for the invention, mention may be made of carrying out disulfide bridge created between two cysteine residues present in a sequence of a peptide to be cyclized.

A disulfide bridge may be created by any known techniques in the art such as for example chemical oxidation.

Cystein residues to be implemented in a disulfide bridge for peptide cyclization may naturally be present in the sequence of said peptide or may be introduced by any known techniques as for example recombinant DNA techniques or chemical synthesis.

As example of cyclized peptide in accordance with the invention, mention may be made of a cyclized peptide set forth as SEQ ID NO:12 (FIG. 8), wherein the asterisk below the cystein residues (C in single letter code) indicates the cystein residues engaged into the disulfide bridge.

A cyclized peptide in accordance to the invention may comprise from 24 to 40 amino acid residues, in particular from 26 to 32 amino-acid residues, and more particularly form 28 to 30 amino-acid residues.

The ability of the antibody of the invention or a fragment thereof or a cyclized peptide of the invention to neutralize HIV may be evaluated through the inhibition of transcytosis assay and the blocking of CD4+ T cells infection assay as described below and illustrated by the examples.

Neutralizing Activity of the Antibody

The neutralizing activity of the antibody of the invention, or fragment thereof, or a cyclized peptide of the invention against HIV may be evaluated by the inhibition of HIV transcytosis across epithelial cells and/or inhibition of HIV infection of CD4+ T cells.

According to an embodiment, the neutralized HIV is more particularly a HIV-1 strain.

The evaluation of ability of a monoclonal antibody or a cyclized peptide of the invention to inhibit the transcytosis of HIV across cells may be performed on any polarized cells. In one embodiment, the polarized cells may be epithelial cells, such as for example the intestinal cell line HT-29 or the endometrial cell line HEC-1 or cells from a human mucosal biopsy (Bomsel et al., Immunity, 1998, 3:277). Typically, the cell lines are grown as a tight polarized monolayer on a permeable filter support (having for example 0.45 μm pore size) forming the interface between two independent chambers, the upper one bathing the apical surface of the epithelial monolayer and the lower one bathing the basolateral surface or the biopsies are mounted in using chambers.

Transcytosis may be initiated by contacting HIV-infected cells, as for example HIV-1-infected peripheral blood mononuclear cells (PBMC), at the apical chamber.

An antibody to be tested may be applied at the apical chamber before or after the application of the HIV infected cells or may be preincubated with the virus HIV-1 or HIV-1 infected cells.

The antibody of the invention to be tested may be applied at various concentrations, for example from about 0.01 to about 10 ng/ml, in particular from about 0.1 to about 5 ng/ml or more preferably from about 0.5 to about 1 ng/ml.

A cyclized peptide of the invention to be tested may be applied at various concentrations, for example from about 0.01 to about 100 μM, in particular from about 0.1 to about 30 μM, and more particularly from about 1 to about 10 μM.

The evaluation of the virus transcytosis, and possibly its inhibition, may be carried out by the detection of HIV nucleic acid or protein in the basolateral medium by any known techniques in the art, such as PCR, RT-PCR, or ELISA.

In one embodiment, a HIV peptide that may be used for the evaluation of the virus transcytosis may be the p24 peptide that may be detected by means of an ELISA assay using, for example, a kit provided by PASTEUR-SANOFI (FRANCE).

In a particular embodiment, when added to about 10⁶ HIV-1 infected PBMCs at a concentration of about 0.5 ng/ml to about 5 ng/ml and incubated for about 1 hour at about 17° C. or at about 4° C. before inoculation of the infected cells to the apical chamber, a monoclonal antibody of the invention or a fragment thereof, may inhibit the virus transcytosis initiated by the addition of 10⁶ HIV-1⁺ PBMC to the apical chamber by at least about 50%, in particular by at least about 75% and more particularly by at least about 95% relative to the virus transcytosis performed without antibody.

In another embodiment, an antibody of the invention, or fragment thereof, may be added to the apical chamber prior to the addition of the HIV-infected PBMCs. In such embodiment, the inhibition of the transcytosis may be at least of about 50% relative to the virus transcytosis performed without antibody.

As example, the Fab and Fd of clone 43, which are also objects of the instant invention, may inhibit the HIV transcytosis by respectively at least about 70% and about 80%.

In another exemplary embodiment, when added to about 10⁶ HIV-1 infected PBMCs at a concentration of about 0,1 to about 100 μM and incubated for about 30 mn at about 17° C. before inoculation of the infected cells to the apical chamber, a cyclized peptide of the invention may inhibit the virus transcytosis initiated by the addition of 10⁶ HIV-1^{+PBMC} to the apical chamber by at least 30%, in particular by at least about 60%, and more particularly by at least about 85% relative to the virus transcytosis performed without a cyclized peptide.

In a particular embodiment, Fab and Fd of clone 43, which are also objects of the instant invention, are able to bind specifically to gp41 expressed at the surface of HIV-1 (JRCSF clone R5) infected PBMCs as measured by flow cytometry analysis, according to conventional methods known in the field.

In another embodiment, the monoclonal antibodies according to the invention, or fragments thereof, may display the ability to inhibit the HIV-infection of CD4+ T cells.

In a particular embodiment, after incubation of the virus at about 1 ng/ml for about 30 min at about 37° C. with the antibody of the invention or fragment thereof, the infection of CD4+ T cells by HIV may be inhibited by at least about 75%, and more particularly by at least about 95% relative to the HIV-infection of CD4+ T cells performed without antibody or with a non specific antibody that does not recognize the virus.

As an example, the Fab clone 43 which is an object of the instant invention may inhibit the HIV-infection of CD4+ T cells by at least 95%.

Nucleic Acids, Vectors and Host Cells

An antibody of the invention has been identified by screening a Fab IgA phage display library obtained as described in the experimental section.

The combinatorial library production and manipulation methods have been extensively described in the literature and are from general knowledge of the skilled person in the art.

The combinatorial Fab phage display library has been screened on peptide P1 having the sequence set forth as SEQ ID NO:7 by means of an iterative panning on antigen immobilized on a solid support (such as enzyme-linked immunosorbent assay (ELISA) plates), method known as micropanning, described in Azzay & Highsmith (Clinical Biochemistry, 2002, 35:425-445).

A starting concentration may be for example about 125 microM, at which the peptide P1 adopts a dimer/tetramer oligomerization state.

The detection of the interaction between the antigen, namely the peptide P1, and the antigen-binding domain displayed on the outside of the bacteriophages may be evaluated by any known techniques in the field. For example, an ELISA assay may be used wherein the peptide P1 is coated in wells of a plate and then phages from the library are applied.

The genes encoding the antigen-binding site, which are unique to each phage, may then be recovered from the phage nucleic acid of the selected phage, sequenced and used to construct genes for a complete antibody molecule or analogue thereof, such as Fab fragment, F(ab′)₂, or scFv as above-described.

The sequence of the genes encoding the antigen-binding site recovered from the selected phage may be sequenced according to any known technique from the skilled person in the art.

For example, sequencing may be carried out by using an automated DNA sequencer with a Taq fluorescent dideoxynucleotid terminator cycle sequencing kit (Applied Biosystems). Double stranded DNA may be prepared, for example, from bacteria and sequencing may be carried using a set of primers that anneal specifically to the vector used for cloning of the H and L chains of Fab, up and down stream of each Fab chain. For example, when using the pComb3X vector, the primers having the sequences set forth in the Table II may be used.

Therefore, according to another embodiment, the present invention also relates to nucleic acid molecules such as cDNA, RNA, and the like encoding the heavy and light chain variable region amino acid sequences of the invention where these sequences are set forth as, respectively, SEQ ID NO:8 and SEQ ID NO:9.

In particular, the nucleic acid sequences of the invention may be for the H chain variable region the sequence set forth as SEQ ID NO:10, and for the L chain variable region the sequence set forth as SEQ ID NO:11.

Of course, due to the degeneracy of the genetic code, variations may be contemplated in the nucleic acid sequence of the heavy and light chain variable region respectively shown in SEQ ID NO:10 and SEQ ID NO:11 which will result in nucleic acid sequences that may be capable of directing production of antibodies or functionally analogues thereof comprising the heavy chain variable region amino acid sequence shown in SEQ ID NO:8 and the light chain variable region amino acid sequence shown in SEQ ID NO:9.

According to an embodiment, the invention also relates to a nucleic acid sequence encoding an antibody of the invention or a fragment thereof or a sequence of a cyclized peptide of the invention. This nucleic acid may be RNA, cDNA and the like. The nucleic acid sequence of the invention may be fused to other nucleic acid sequences to construct chimeric proteins by any known molecular biological techniques in the art. For example, the nucleic acid sequence of the invention may be fused to nucleic acid encoding for His-Tag of HA-Tag, which may be used thereafter, for example, to help to purify the antibodies of the invention.

According to another embodiment, the present invention also relates to an expression vector comprising a nucleic acid sequence encoding a monoclonal antibody in accordance with the invention, or a fragment thereof or a sequence of a cyclized peptide of the invention.

Such an expression vector may be used for the expression of a monoclonal antibody according to the invention, or fragment thereof or a sequence of a cyclized peptide of the invention, in various types of cells. Such a vector may be a plasmid or a viral vector. Such a vector may be capable of autonomous replication in a host cell, or may be integrated into the genome of a host cell.

As example of expression vectors that may convene for achieving the invention, mention may be made of pComb3X or pASK88.

The instant invention also contemplates the expression of an antibody of the invention or fragment thereof or a cyclized peptide of the invention by means of various host cells, such as bacteria, mammal cells (CHO, or HEK293), insect cells (such as Sf9 cells), or plant cells (tobacco, tomatoes).

After expression, an antibody of the invention may be isolated and purified by any known techniques in the field. For example, an antibody may be expressed with a His-Tag or a HA-Tag, and may be thereafter purified on a Nickel column or with a specific antibody as usually performed in the art.

After expression of a linear sequence of a cyclized peptide of the invention, a step of cyclization to obtain a loop structure may be performed by any known techniques in the art, as previously indicated.

Therefore, the present invention is also directed to a host cell transformed with a nucleic acid sequence encoding for a monoclonal antibody in accordance with the invention or a fragment thereof or a sequence of cyclized peptide of the invention. The host cell may be obtained according to any known technique of transformation known in the art, such as electroporation, calcium phosphate techniques, lipofection, infection with, for example, a recombinant virus. The present invention is directed to a host cell transformed with a nucleic acid sequence encoding for a monoclonal antibody in accordance with the invention or a fragment thereof or a sequence of cyclized peptide of the invention, such as electroporation in the case of bacteria, or lipofection in the case of mammal cells (CHO).

Pharmaceutical Composition

According to one embodiment, the invention is related to a pharmaceutical composition comprising as active agent an effective amount of at least one agent chosen among an antibody of the invention, or a fragment thereof, in particular the clone 43, a H chain variable region of the invention, a recombinant anti-HIV antibody of the invention or a fragment thereof, a cyclized peptide of the invention, a nucleic acid according to the invention, an expression vector according to the invention or a host cell according to the invention, and a suitable carrier.

According to another embodiment, the above-described active agent may be used for manufacturing a medicament intended to be used in the prophylaxis and/or the treatment of HIV-infection, and in particular of HIV-1 infection.

The term “effective amount” means the minimal amount necessary to observe the expected effect, i.e. a neutralization of HIV and/or prevention of HIV infection. A therapeutically or prophylactic effective amount of an antibody according to the invention, or a fragment thereof, for individual patient may be determined as being the amount of antibody given to the individual to arrive at the therapeutic or prophylactic effect, i.e. reduction or prevention of the infection, while minimizing side effects. The effective amount may be measured by serological decreases in the amount of HIV antigens in the individual.

An exemplary, non limiting range for a therapeutically or prophylactically effective amount of an antibody of the invention, or a fragment thereof, or a cyclized peptide of the invention, is of about 0.1-100 mg/kg, more particularly about 0.5-50 mg/kg, more particularly about 1-20 mg/kg and even more particularly about 1-10 mg/kg.

According to another embodiment, a monoclonal antibody of the invention, or fragments thereof, or a cyclized peptide of the invention, or a pharmaceutical composition of the invention may be used in passive immunotherapy, by administering to an individual liable to be infected or susceptible to be exposed to HIV, a therapeutically effective amount of at least an antibody according to the invention or a fragment thereof, or a cyclized peptide of the invention, or a pharmaceutical composition of the invention. The passive immunotherapy of the invention may be practiced on individuals exhibiting AIDS or related conditions caused by HIV infections or individuals at risk of HIV infections.

According to one embodiment, the passive immunotherapy of the invention may also be practiced prophylactically, i.e. before a susceptible exposure to HIV.

The pharmaceutical composition of the invention may be administered per os, parenterally (intra-veinously or intra-nasaly or the like), topically, rectally, vaginally or the like.

Therefore, the pharmaceutical composition of the invention may be prepared under various galenic forms, such as injectable or infusible sterile solution, dispersion or suspension, tablet, pills, powders, liposomes, suppositories and cream.

The suitable carrier to be used in the manufacture of the pharmaceutical composition of the invention is to be adapted according to the galenic form intended to be carried out. Such suitable carriers include, but are not limited to, water, saline, phosphate buffered saline, dextrose, glycerol and the like, as well as combinations thereof. Pharmaceutically acceptable substances such as wetting or emulsifying agents, preservatives or buffers may also be included in the pharmaceutical compositions of the invention.

The pharmaceutical composition of the invention may also include, in addition of the active agents of the invention, at least one other active agent against HIV, such as antiretroviral drugs. According to another embodiment, such additional antiretroviral drugs may be administered in a combination with the pharmaceutical composition of the invention, simultaneously, separately, or sequentially in time.

Within another embodiment, an antibody of the invention, or fragment thereof, or cyclized peptide of the invention, may also be labelled for therapeutic purpose, in which case the labelling agent may be a drug conjugate or a toxin, such as a radioisotope or a radionuclide (¹³¹I, ⁹⁹Tc, ¹¹¹In or the like), pertussis toxin, taxol, cytochalasin B, doxorubicin and the like.

Diagnostic Composition and Application

A monoclonal antibody of the invention or a fragment thereof, or cyclized peptide of the invention may also be used as a diagnostic agent in a method for detecting in vitro a HIV strain, and in particular a HIV-1 strain, in a sample.

In one of its embodiment, a method of the invention may comprise at least a step of:

a) contacting the sample with at least an antibody of the invention or a fragment thereof, or a cyclized peptide of the invention, under conditions suitable to form a complex between said antibody, or fragment thereof, or between said cyclized peptide, and a HIV protein comprising a peptide having the sequence set forth as SEQ ID NO:3, or a functional analogue thereof, and

b) detecting the presence of said complex.

The detection of the complex may be carried out by any immunoassay known in the art.

Such assays include, but are not limited to, radioimmunoassay, Western blot assay, immunofluorescent assay, enzyme immunoassay (such as ELISA), chemiluminescent assay, immunohistochemical assay and the like.

Additionally for the ease of detection, a monoclonal antibody of the invention, or a fragment thereof, or a cyclized peptide of the invention may be labelled by means of a detectable marker. As example of labelling agent, mention may be made of fluorescent compounds, such as fluorescein or rhodamine; enzymes such as horseradish peroxydase, beta-galactosidase or luciferase; biotin allowing detection through indirect measurement of avidin or streptavidin binding; or radiolabelled amino acid comprising, for example, as radioisotope or radionuclide ³H, ¹⁴C or ¹²⁵I.

According to another of its embodiments, the instant invention is also directed to a diagnostic composition comprising an antibody in accordance with the invention or a fragment thereof or a cyclized peptide

In order that this invention may be better understood, the following examples are set forth.

These examples are for purposes of illustration only and are not to be construed as limiting the scope of the invention in any manner.

### LEGENDS OF THE FIGURES

FIG. 1: It represents immunoblot of gp41-derived HIV-protein exposed to anti-HIV-1 IgA-content cervico-vaginal secretion of Highly Exposed Persistently IgG Seronegative (HEPS) individuals. As a positive control, immunoblot of gp41 peptide was performed with serum obtained from HIV seropositive individuals. And as negative control, the same experiment was performed with serum of HIV seronegative individuals.

FIG. 2: It represents a schematic view of the plasmid pComb3X wherein the variable and constant regions of the heavy and light chains from the Fab IgA phage library have been inserted, respectively, between restriction enzyme sites Sac 1 and Xba 1, and between Xho 1 and Spe 1.

FIG. 3: It represents the inhibition of HIV-1 transcytosis across the endometrial HEC-1 cell line by selected IgA Fab clones. Negative control was obtained by performing the experiment with a non specific Fab clone that did not recognize P1 in ELISA. Positive control was achieved by using the 2F5 antibody IgA.

FIG. 4: It represents the inhibition of infection of HeLa CD4+ CXCR4+ LTR LacZ cells by HIV-1 Lai2 preincubated with selected Fabs at a concentration of 1 ng/ml for 30 min at 37° C. before incubation for 1 h hour at 37° C. with the cells. Negative control (standard) was performed using no Fabs or non specific Fab clone that did not recognize P1 in ELISA. Positive control was performed using the 2F5 antibody IgA.

FIG. 5: It represents the amino acid sequence of the complementarity determining region 3 (CDR3) (SEQ ID NO:1) as well as the CDR1 and CDR2 (SEQ ID NO:2 and SEQ ID NO:3) of a H chain variable region of an antibody according to the invention. Also are represented the amino acid sequences of the complementarity determining regions CDR1, CDR2 and CDR3 set forth as SEQ ID NO:4, SEQ ID NO:5 and SEQ ID NO:6 of a L chain variable region of an antibody according to the invention. Also is represented the amino acid sequence SEQ ID NO:7 of the peptide P1 corresponding to the amino acid sequence comprised between position 649 and 684 of gp41 of HIV-1.

FIGS. 6A and 6B: It represents the amino acid sequence and the nucleic acids sequence of the heavy chain variable region (SEQ ID NO:8 and SEQ ID NO:10) and of the light chain variable region (SEQ ID NO:9 and SEQ ID NO:11) of the clone Fab 43.

FIG. 7: It represents the amino acid sequences of the frame work region (FR) and complementarity determining region (CDR) of the light and heavy chains of the Fab IgA clone 43 obtained using the IMTG program available free on line (and created by M. P. Lefranc, France). IMGT/V-QUEST software (http://imgt.cines.fr)

FIG. 8: It represents the amino acid sequences of a cyclized peptide comprising a complementarity determining region 3 and being cyclized by a disulfide bridge between two cystein residues (SEQ ID NO:12). The cystein residues engaged in the disulfide bridge are identified by an asterisk.

FIG. 9: It represents the inhibition of HIV-1 transcytosis across the endometrial HEC-1 cell line by a cyclized peptide of sequence as set forth as SEQ ID NO:12. Negative control was obtained by performing the experiment with a linear peptide.

Table I: It represents the sequences of the primers used to amplify the DNA obtained from the HEPS individuals.

Table II: It represents the sequences of the primers used to identify and sequence the nucleic acids sequences of Fab IgA from the phage display library.

### EXAMPLE 1

Identification of Anti-HIV-1 IgA in Cervico-Vaginal Secretions of Highly Exposed Persistently IgG Seronegative (HEPS) Individuals.

Cervical secretions from 56 HEPS originated from Cambodia were tested for HIV-envelope glyco-protein S-IgA. Secretions were collected after two days of sexual abstinence using 3 ml of sterile PBS. Samples were centrifuged and frozen and stored at −80° C. Sample contamination by sperm was measured using the seminal fluid detection assay (SEMA; HUMANGEN FERTILITY DIAGNOSTICS, Charlottesville, Va.) according to the manufacturer but optimized by increasing the incubation times and using O-phenylenediamine dihydrochloride as substrate. Contaminated samples were discarded.

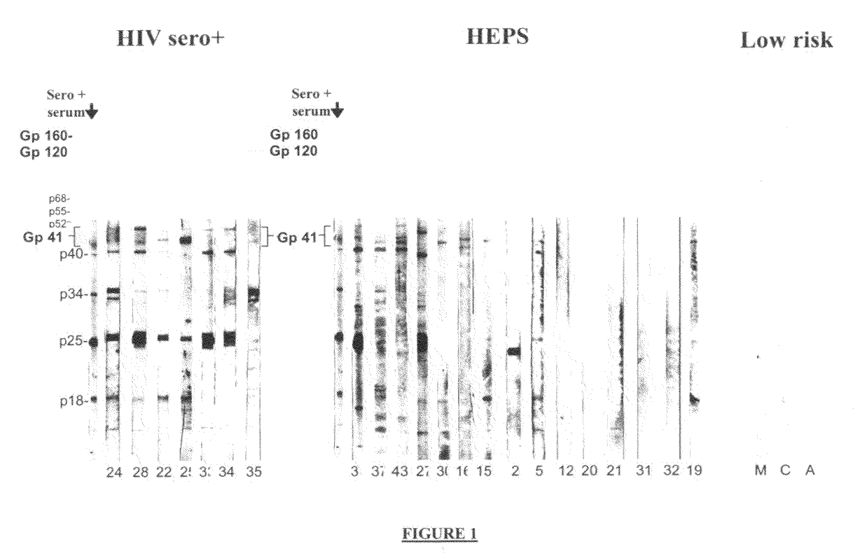

The presence of specific IgA antibodies to HIV was tested by western blot using a commercially available kit (New Lav Blot1, Sanofi-Pasteur, France) using procedure according to the manufacturer. Among the tested samples, 22 contained a high level of anti-HIV envelope gp41 Secretory IgA (S-IgA) (FIG. 1).

### EXAMPLE 2

Construction of a Combinatorial Phage Display Library Expressing Fab of IgA

Mucosal B cells of the 22 identified HEPS of the example 1 were used to construct a combinatorial phage display library expressing Fab of IgA.

Total RNA was prepared from an enriched B-cells population form cervico-vaginal secretions of the 22 HEPS individuals using standard techniques. Total RNA (60 μg) was purified from cervical B cells by a rapid single step guanidinium isothiocyanate/phenol chloroform-based RNA isolation (Chomczynski & Sacchi, Anal. Biochem., 1987, 162:156-9.). This total RNA was transformed in complementary DNA (cDNA) using reverse transcriptase-polymerase chain reaction (RT-PCR). The obtained cDNA library was amplified using mucosal Fab IgA specific primers set forth in Table I.

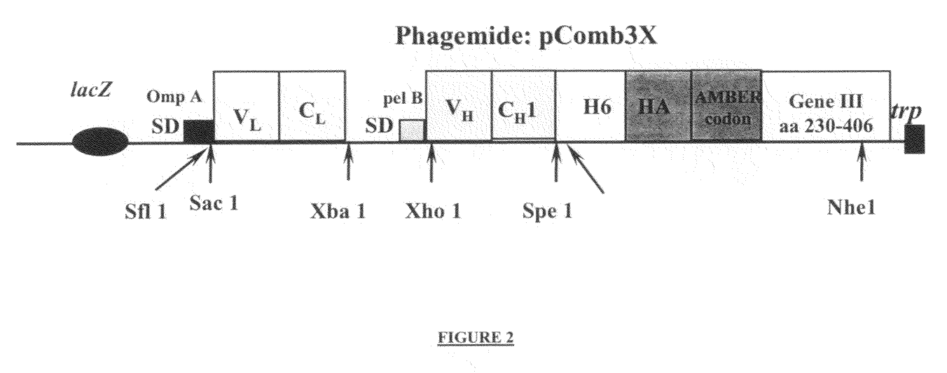

The amplified DNA was then digested with Sac I and Xba 1 for light chain (L) and Xho 1 and Spe 1 for heavy chain (H), and the digested products were inserted in the vector pComb3X (Barbas et al., Proc Natl Acad Sci USA, 1991, 88:7978-82) digested with the same enzymes. The digested vector and the digested PCR product were ligated using DNA ligase of phage T4 (4 inserts plus 1 vector plus T4DNA ligase, overnight at 14° C.) (FIG. 2). The obtained recombinant plasmids were then used to transform bacteria E.coli (pilus+, male) TG1 by means of electroporation.

The library of antibody cDNAs in the culture after transformation of the bacteria was then expressed on bacteriophage by super infection with the helper phage VCS-M13 (10¹² pfu) (Stratagene, La Jolla, Calif.)

The phage preparations were harvested. Phages were precipitated by addition of 20% (wt/vol) polyethylene glycol 8000 and 2,5 M NaCl followed by incubation on ice for one hour. After centrifugation, phage pellet was resuspended in phosphate-buffered saline (PBS) and microcentrifuged for few minutes to pellet debris.

Restriction digest analysis from individual colonies indicated that approximately 35% of the phages contain Fab IgA and that the library contained titers were typically close to 10⁷ cfu/ml, and 10⁷ different clones.

### EXAMPLE 3

Screening of the Fab IgA Phage Library on Peptide P1 (SEQ ID NO:7)

The Fab IgA phage display library has been screened by means of micro panning, in which the antigen concentration was the varying condition at each round. The antigen used for the screening was peptide P1 (Alfsen & Bomsel, 2002, J. Biol. Chem., 277:25649-59; obtained from Eurogentec, Belgium). The starting amount of peptide P1 used was 20 μg, and the amount was divided by 2 at each round to reach finally the amount of 2.5 μg, at the fourth.

Wells of a 96-plates (Exiqon peptide Immobilirez, 10202-111-10) were coated with 125 microM peptide P1, concentration at which it is dimeric, then blocked with BSA for 2 hours at 37° C. The concentrated phages prepared from the library were then applied to the wells at 10¹²-10¹³ pfu, for 2 hours at 37° C. Unbound phage were then removed by vigorous washing, for the first and second rounds of selection, 10 times with PBS containing 0.1% Tween-20, then 10 times with PBS to remove the detergent. For the subsequent rounds of selection washing was carried out 20 times with PBS containing 0.1% Tween-20, then 20 times with PBS. The specific elution of phages bearing epitopes of P1-binding surface Fabs was performed by treatment with 0.1 M glycine-HCL, adjusted to pH 2.2 for 10 min. The eluate was immediately neutralized and used to infect 2 ml of fresh E. coli TGI bacterial culture (OD₆₀₀=1). Bacteria were incubated at 30° C. for 30 minutes, culture volume was increased and culture was incubated in at 37° C. shaker for 1 hr. and thereafter 10¹² pfu/ml of VCS-M13 helper phage was added for overnight production of recombinant phages. The eluted phages were amplified between each panning round.

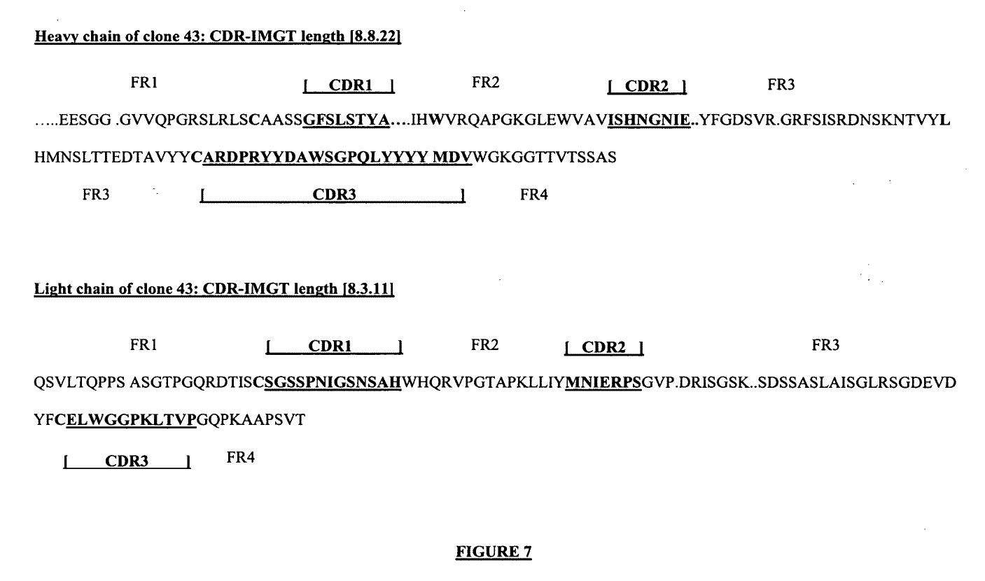

After panning, individual clones from the four rounds were grown and the presence of Fab was monitored by PCR using specific primers set forth in Table II which hybridize to the vector upstream and downstream to the Fab light and heavy chains. Clone containing Fab IgA were converted to soluble Fab by transforming in an E. coli amber non-suppressor strain and sequenced to determine the variable region sequences of Heavy (H) and Light (L) chains using IMGT/V-QUEST software (FIG. 7).

Sequencing of Immunoglobuline Genes

Sequencing was done using an automated DNA sequencer with a Taq fluorescent dideoxynucleotid terminator cycle sequencing kit (Applied Biosystems). Double stranded DNA was prepared from bacteria and sequencing was carried out using a set of primers (see Table II) that anneal specifically to the pComb3X vector up and down stream of each Fab chain.

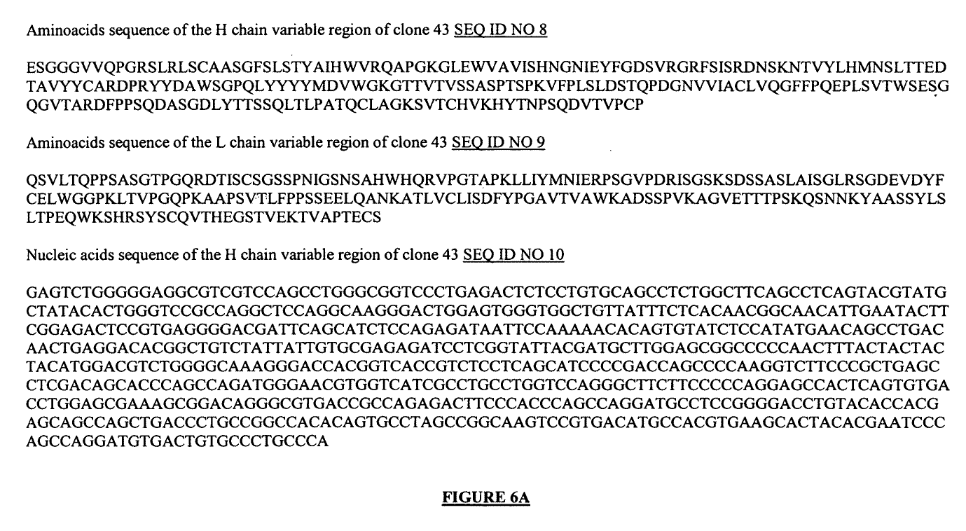

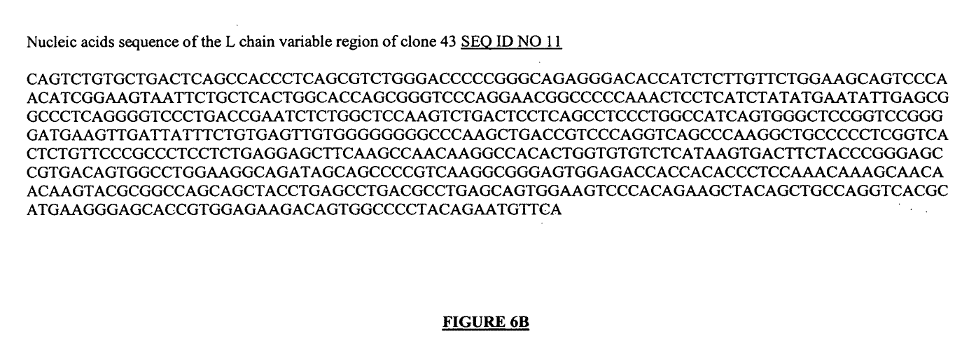

FIG. 6 shows the result of the sequencing of the H and L chain variable regions of clone 43 (SEQ ID NO:10 and SEQ ID NO:11).

FIG. 7 shows the identification in the corresponding amino acid sequences of the complementarity determining regions using the IMGT software.

The vector used for cloning has an amber codon between the tag (His-tag and HA-tag) sequences and the phage protein pIII providing the opportunity to either produce the antibody fused with the coat protein of the phage, using a E. coli suppressor strain such as TG1, or to produce soluble antibodies by expressing the vector in a non-suppressor strain, such as TOP10. Taking into account this advantage, DNA of phages was used to transform TOP-10 E. coli (amber nonsupressor-strain) in order to express soluble Fab fragments without the pIII fusion protein. Fab expression was induced using 1 M Isopropyl b-D-thiogalactopyranoside (IPTG)

For large-scale expression of the monoclonal antibody of the invention the whole operon was transferred from the pComb3X to the vector pASK88 (Skerra A., Gene, 1994, 151 (1-2): p. 131-5; Skerra A., Gene, 1994, 141(1): p. 79-84; Skerra A., et al., Biotechnology (New York), 1991. 9(3), p. 273-8; Skerra A. & A. Pluckthun, Protein Eng, 1991, 4(8): p. 971-9.). Fab DNA of the monoclonal antibody of the invention was amplified with specific primers introducing restriction sites needed for subcloning into the pASK88 vector. The amplification products were purified by agarose gel electrophoresis and cut with the restriction enzymes PstI and NcoI in the case of heavy chain and SacI and HindIII in the case of light chain. The Fd (VH-CH1) and light chain (VL-CL) genes were separately inserted and ligated into pASK88 digested with the same restriction enzymes using standard protocols. The ligations were transformed into thermocompetent JM83 (provided by Dr. Skerra) bacterial cells and plated for separation in individual clones. Plasmid DNA for several clones was analyzed by restriction digest and, for some of them, by double strand sequencing using specific primers.

The expression vector pASK88 was designed for the convenient cloning of immunoglobulin variable domain genes as well as periplasmic secretion of the corresponding Fabs fragment in Escherichia coli. On this plasmid, expression was under control of the tetracyclin promoter.

Large quantities of the monoclonal antibody of the invention were obtained using this vector. Preparative expression was performed on the 1-litre scale, employing E. coli K-12 JM83 as expression host. The cells were grown to a mid-log phase, and the Fab expression was then induced by 0.2 mg/L anhydrotétracycline for 4 h or overnight.

Purification of the monoclonal antibody of the invention was performed on Ni-NTA Spin Columns (Qiagen) by interaction with the His-tag. The periplasmic fraction filter sterilized was applied on the column and a gradient of 250-500 mM imidazole in chromatography buffer was applied to collect the samples.

### EXAMPLE 4

ELISA Assay Performed with Soluble IgA Fab Clones from the Phage Display Library.

Elisa was performed with soluble IgA Fab obtained from TOP10F′ bacteria (pComb3x system) and with soluble IgA Fab obtained from K-12 JM83 bacteria (pASK88 system).

Wells of plates (Exiqon peptide Immobilized, 10202-111-10 or NUNC, 439454) were coated with the P1 peptide (amino acid sequence 649-684 of gp41 of HIV-1) and blocked with BSA for 2 hours at 37° C. Then, the purified IgA at 2 ng/ml were added and incubated for 2 hours at 37° C. Detection was done by mouse anti-Ha antibody (clone 12CA5, Roche) or anti-His (PentaHis, Qiagen 34660) followed by horseradish peroxydase goat anti-mouse antibody (Caltag Laboratories, H1003). The enzymatic reaction was developed by adding TMB (3,3′,5,5′-tetramethyl-benzidine, Kikergaard & Perry Laboratories Inc.) as a substrate and absorbance was measured at 450 nm after addition of 1M acid phosphoric using an ELISA reader. The positive control was 2F5 antibody and the negative control was a clone obtained after panning that not recognizes P1 in ELISA.

### EXAMPLE 5

Inhibition of Transcytosis

HIV-1 transcytosis across epithelial cells and neutralisation of transcytosis by antibodies were performed on intestinal cell line HEC-1 grown as a tight, polarized monolayer for 7 days on a permeable filter support (0.45 μm pore size) forming the interface between two independent chambers, the upper one bathing the apical (luminal) surface of the epithelial monolayer and the lower one bathing the basolateral (serosal) surface. PBMC were obtained and prepared as described in Lagaye et al., (J. Virol, 2001, 75:4780). Then PBMCs were activated with phytohemagglutinin (PhA) for 48 h and inoculated with HIV-1 JRCSF clone R5 or YU2 and used at day 7 post infection. Purified S-IgA (5 ng/ml) were added to the apical chamber and incubated for 10 min at 37° C. To initiate virus transcytosis, 2.10⁶ HIV-1⁺ PBMC were added to the apical chamber. Contact between HIV-1⁺ PBMC and the epithelial cell monolayer resulted in rapid budding of the HIV-1 virions, followed by their transcytosis from the apical to the basolateral pole of the epithelial cells. After 2 hours, inhibition of transcytosis by antibody was determined by detection of the protein p24 of HIV in the basolateral medium by ELISA (Coulter, France or PASTEUR SANOFI, FRANCE). The level of p24 in the absence of antibody or in the presence of a P1 non specific Fab or in the presence of the control IgA 2F5 was measured as negative and positive control respectively with a value of 100, 98, and 35% respectively. The value of the negative control was taken as 100% of transcytosis and used to express the results.

The experiments were performed in three independent experiments.

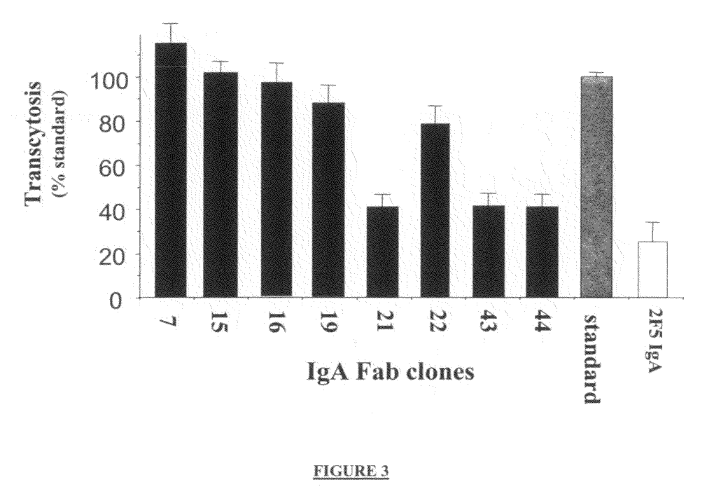

The results allow identifying 3 clones that were able to inhibit the HIV transcytosis by more than 50% among which are clones 43 and 44 (FIG. 3).

### EXAMPLE 6

Inhibition of HIV Infection

HIV-1 Lai2 was preincubated with selected Fabs at a concentration of 1 ng/ml for 30 min at 37° C. before incubation with HeLa CD4+ CXCR4+ LTR LacZ cells (Dragic et al., 1992, J. Virol., 66:4794-4802) for 1 hour at 37° C. Unbound virus was removed by washing cells two times before incubation for 36 h at 37° C. Cells were then analyzed for HIV content by measuring beta-galactosidase activity in the cell extract using the CPRG substrate and colorimetric measurement. Negative control was performed using no Fabs or non specific Fab clone that did not recognize P1 in ELISA.

Results on FIG. 3 are mean of at least two independent experiments each performed in duplicate.

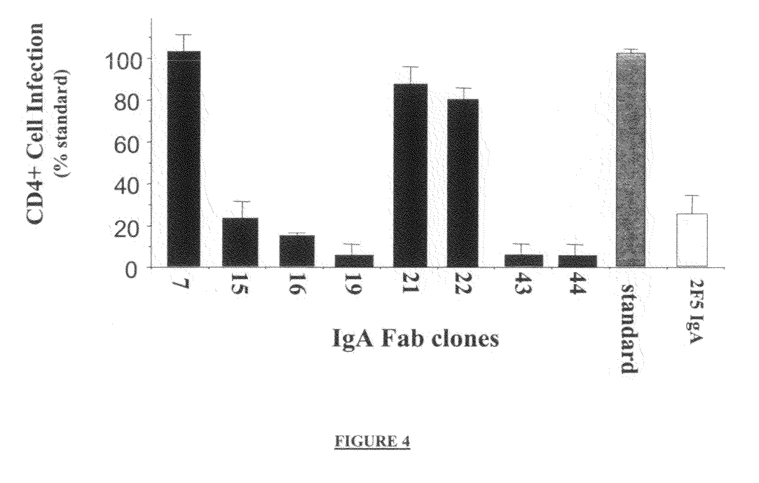

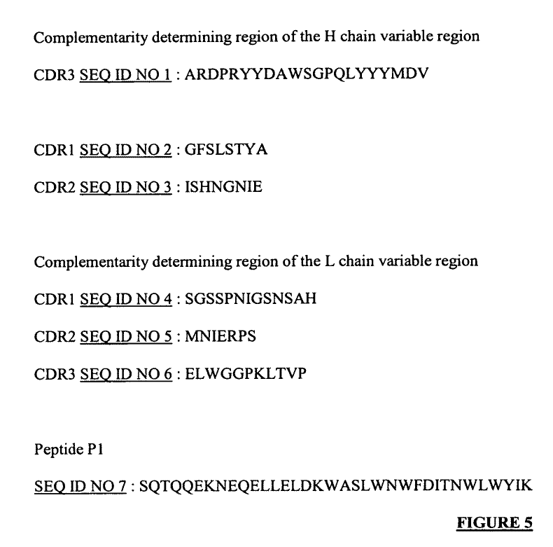

The results have allowed the identifying of 5 clones that were able to inhibit the HIV-1 infection of CD4+ cells by more than 75% or more than 95% (FIG. 4). Among those clones, 2 were also able to inhibit HIV transcytosis which are clones 43 and 44 (see example 4).

### EXAMPLE 7

Preparation of a Cyclized Peptide Comprising a CDR3 of Sequence set Forth as SEQ ID NO:1

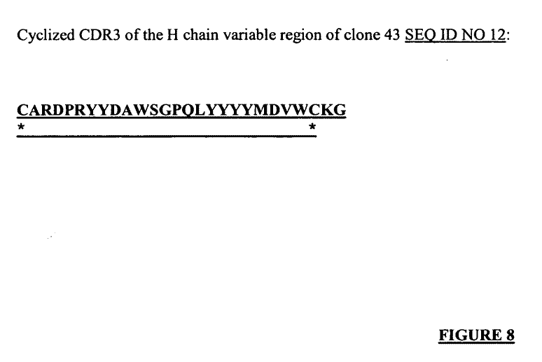

The amino acid sequence of the CDR3 derived from Fab clone 43 with the surrounding amino acid residues cysteine in N-terminal position and the sequence WGKG at the C-terminal end has been modified by substitution of the glycin residue in the sequence WGK with a cystein residue (see FIG. 8, SEQ ID NO:12).

The cyclization of the peptide was performed under oxidative conditions as described in LEVI et al., (Proc. Natl Acad, Sci USA, 1993, 90:4374). Additionally, the cyclized peptide has been biotinylated.

Inhibition of Transcytosis with Cyclized CDR3

The HIV-1 transcytosis inhibition protocol was performed as described in example 5, except that HIV-1 R5 strains from clades C and D were used.

Before inoculation of the PBMCs with HIV-1 R5 strains from clades B or C, cells were preincubated for 30 minutes at 17° C. with a cyclized CDR3 (obtained as previously indicated) or a linear CDR3 as a control.

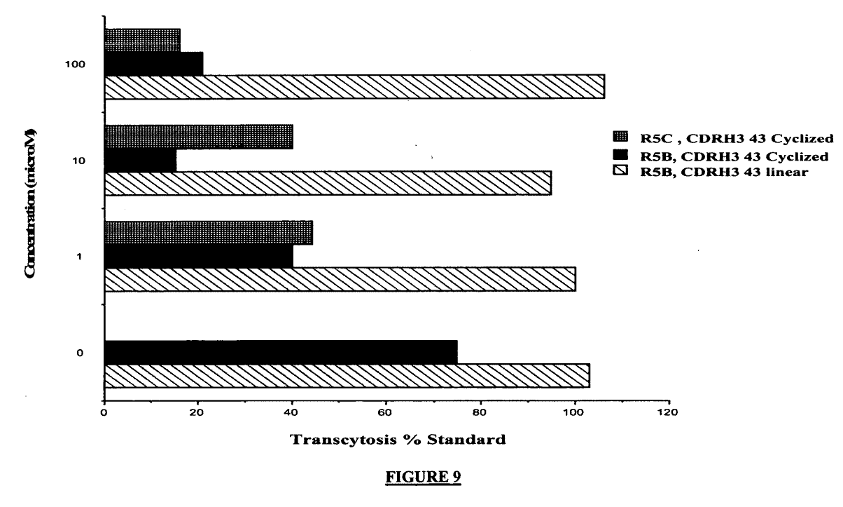

The results illustrated on FIG. 9 indicate that the cyclized CDR3 derived from Fab clone 43 was efficiently able to inhibit the transcytosis by more than 50%.

TABLE I

Primers used for the amplification of human α,
(Fd) and K and λ sequences α1

V_{H} 5′ primers (5′-3′)

| | | |
|---|---|---|
| V_{H1a} | CAG GTG CAG CTC GAG CAG TCT GGG | (SEQ ID NO:13) |
| V_{H1f} | CAG GTG CAG CTG CTC GAG TCT GGG | (SEQ ID NO:14) |
| V_{H2} | CAG GTC ACC TTG CTC GAG TCT GGT | (SEQ ID NO:15) |
| V_{H3a} | GAG GTG CAG CTC GAG GAG TCT GGG | (SEQ ID NO:16) |
| V_{H3f} | GAG GTG CAG CTG CTC GAG TCT GGG | (SEQ ID NO:17) |
| V_{H4f} | CAG GTG CAG CTG CTC GAG TCG GG | (SEQ ID NO:18) |
| V_{H4g} | CAG GTG CAG CTA CTC GAG TGG GG | (SEQ ID NO:19) |
| V_{H5} | GAG GTG CAG CTC GAG CAG TCT GG | (SEQ ID NO:20) |
| V_{H6} | CAG GTA CAG CTC GAG CAG TCA GG | (SEQ ID NO:21) |
| V_{H7} | CAG GTG CAG CTC GAG CAA TCT GG | (SEQ ID NO:22) |

C_{H} 3′ primers (5′-3′)

| | | |
|---|---|---|
| CHA-1 | AGT TGA ACT AGT TGG GCA GGG CAC AGT CAC | (SEQ ID NO:23) |
| CHA-2 | AGT TGA ACT AGT TCG GCA GGG AAC AGT CAC | (SEQ ID NO:24) |

V_{K} 5′ primers

| | | |
|---|---|---|
| V_{K1a} | GAC ATC GAG CTC ACC CAG TCT CCA | (SEQ ID NO:25) |
| V_{K1s} | GAC ATC GAG CTC ACC CAG TCT CC | (SEQ ID NO:26) |
| V_{K2a} | GAT ATT GAG CTC ACT CAG TCT CCA | (SEQ ID NO:27) |
| V_{K2b} | GAT ATT ACC CAG ACT CCA | (SEQ ID NO:28) |
| V_{K3a} | GAA ATT GAG CTC ACG CAG TCT CCA | (SEQ ID NO:29) |
| V_{K3b} | GAA ATT GAG CTC AC(G/A) CAG TCT CCA | (SEQ ID NO:30) |
| V_{K4} | GAC ATC ACC CAG TCT CCA | (SEQ ID NO:31) |
| V_{K5} | GAA ACG GAG CTC ACG CAG TCT CCA | (SEQ ID NO:32) |
| V_{K6} | GAA ATT GAG CTC ACT CAG TCT CCA | (SEQ ID NO:33) |

V_{λ}5′primers

| | | |
|---|---|---|
| V_{L1} | CAG TCT GAG CTC ACG CAG CC(G/A) CCC TC | (SEQ ID NO:34) |
| V_{L2} | CAG TCT GAG CTC ACT CAG CCT GCC TC | (SEQ ID NO:35) |
| V_{L3} | GCC TCC TAT GAG CTC ACT CAG CCA | (SEQ ID NO:36) |
| V_{L4a} | CAG CCT GAG CTC ACT CAA TCA TCC TC | (SEQ ID NO:37) |
| V_{L4b} | CAG CCT GAG CTC ACT CAG CCC CCG TC | (SEQ ID NO:38) |
| V_{L5} | CAG CCT GAG CTC ACT CAG CCG (G/T)CT TCC | (SEQ ID NO:39) |
| V_{L6} | AAT TTT GAG CTC ACT CAG CCC CAC | (SEQ ID NO:40) |
| V_{L7} | CAG ACT GAG CTC ACT CAG GAG CCC | (SEQ ID NO:41) |
| V_{L8} | CAG ACT ACC CAG GAG CCA TCG TTC | (SEQ ID NO:42) |
| V_{L9} | CAG CCT GAG CTC ACT CAG CCA CCT TC | (SEQ ID NO:43) |
| V_{L10} | CAG GCA GAG CTC ACT CAG CCA CCC TCG | (SEQ ID NO:44) |

CL3′ primers
κ3′ primers

| | | |
|---|---|---|
| CLK | TCC TTC TAG ATT ACT AAC ACT CTC CCC TGT TGA AGC | (SEQ ID NO:45) |

λ3′ primers

| | | |
|---|---|---|
| CL2 | CGC CGT CTA GAA TTA TGA ACA TTC TGT AGG | (SEQ ID NO:46) |

TABLE II

Primers used to identify and sequence the
nucleic acid sequences of Fab IgA from the
phage display library

| | | |
|---|---|---|
| OmpA | AAG ACA GCT ATC GCG ATT GCA G | (SEQ ID NO:47) |
| PelSeq | ACC TAT TGC CTA CGG CAG CCG | (SEQ ID NO:48) |
| PelSeq | GAG CAG CTG CAC CTC GGC CAT G | (SEQ ID NO:49) |
| Rev | | |
| Rev | GCC CCC TTA TTA GCG TTT GCC | (SEQ ID NO:50) |
| | ATC | |

1. A monoclonal antibody or a fragment thereof, recognizing a peptide of sequence set forth as SEQ ID NO 7 or an analogue thereof, wherein the complementarity determining region 3 (CDR3) of its H chain variable region comprises the peptide sequence set forth as SEQ ID NO 1 or a functional analogue thereof.

2. The monoclonal antibody or a fragment thereof, according to claim 1 wherein the H chain variable region further comprises at least one CDR selected from the group consisting of CDR1 and CDR2 having respectively the peptide sequence set forth as SEQ ID NO 2 and SEQ ID NO 3 or functional analogues thereof.

3. The monoclonal antibody or a fragment thereof, according to claim 1, wherein it comprises a L chain variable region comprising at least one CDR selected from the group consisting of CDR1, CDR2 and CDR3, having respectively the peptide sequence set forth as SEQ ID NO 4, SEQ ID NO 5, SEQ ID NO 6 or functional analogues thereof.

4. The monoclonal antibody or fragment thereof, according to claim 1, wherein it is an IgA.

5. The monoclonal antibody or fragment thereof, according to claim 1, wherein it is a human antibody.

6. The monoclonal antibody or fragment thereof, according to claim 1, wherein the heavy chain variable region has the amino acid sequence set forth as SEQ ID NO 8 and the light chain variable region has the amino acid sequence set forth as SEQ ID NO 9.

7. The monoclonal antibody or fragment thereof, according to claim 1, wherein it has the ability to neutralize HIV.

8. The monoclonal antibody or fragment thereof, according to claim 7, wherein the neutralized HIV is a HIV-1 strain.

9. A H chain variable region recognizing a peptide sequence set forth as SEQ ID NO 7 or an analogue thereof, wherein the CDR3 of said H chain variable region comprises a peptide sequence set forth as SEQ ID NO 1 or a functional analogue thereof.

10. The H chain variable region according to claim 9, wherein it further comprises at least one CDR selected from the group consisting of CDR1 and CDR2 having respectively the peptide sequence set forth as SEQ ID NO 2 and SEQ ID NO 3 or functional analogues thereof.

11. The H chain variable region according to claim 9 comprised in a recombinant anti-HIV antibody or a fragment thereof.

12. A cyclized peptide having from 24 to 40 amino acid residues and comprising a loop, said loop comprising a peptide of sequence set forth as SEQ ID NO 1, or a functional analogue thereof, said cyclized peptide recognizing a peptide of sequence set forth as SEQ ID NO 7 or a functional analogue thereof.

13. The cyclized peptide according to claim 12 wherein a disulfide bridge is bonding the extremities of said loop.

14. The cyclized peptide according to claim 12, wherein it comprises a peptide sequence set forth as SEQ ID NO 12.

15. A nucleic acid sequence encoding a monoclonal antibody or a fragment thereof, as defined according to claim 1.

16. The nucleic acid sequence according to claim 15 wherein the sequence of the heavy chain variable region is set forth as SEQ ID NO 10 and the sequence of the light chain variable region is set forth as SEQ ID NO 11.

17. The nucleic acid sequence as defined according to claim 15, comprised in an expression vector.

18. The nucleic acid sequence as defined according to claim 15, comprised in a transformed host cell said transformed host cell all being transformed by said nucleic acid sequence.

19. A method for providing passive immunotherapy to an individual liable to be infected with HIV comprising administering to said individual a therapeutically effective amount of at least an antibody or a fragment thereof, as defined according to claim 1.

20. A method for providing passive immunotherapy to an individual liable to be infected with HIV comprising administering to said individual a therapeutically effective amount of at least an antibody or a fragment thereof, as defined according to a cyclized peptide according to claim 12.
